(19) United States
(12) Patent Application Publication
(10) Pub. No.: US 2026/0358314 A1
(43) Pub. Date: Nov. 12, 2026

(54) Gas Detection System

(71) Applicant: WIT, Inc, Houston, TX (US)

(72) Inventor: Rosbel Gonzalez-Perez, Houston, TX (US)

(73) Assignee: WIT Inc., Houston, TX (US)

(21) Appl. No.: 19/671,890

(22) Filed: May 8, 2026

Related U.S. Application Data
(60) Provisional application No. 63/802,001, filed on May 8, 2025.

(51) Int. Cl. G01N 33/00; F16K 24/04; F24F 11/33

(52) U.S. Cl. CPC G01N 33/0032; F16K 24/04; F24F 11/33

(57) ABSTRACT

Described herein is a gas detection system comprising a plurality of detectors that generate response patterns when sensors contact ambient air. The system includes a server having a database storing known response patterns corresponding to known gases, substances, and odors, and an identification module that compares detector response patterns with known patterns to generate an identification classification. The system further includes a validation module that receives the identification classification, validates it according to validation criteria prior to initiating a response action, and generates a validated output only when the criteria are satisfied. A plurality of actuators communicatively coupled to the validation module control response devices upon receiving the validated output. A mobile application provides remote monitoring and control, displaying sensor data, identification results, and validation status to ensure reliable detection and prevent false positive actuations.

### CROSS-REFERENCE TO PRIOR APPLICATION

The present application claims priority and benefit of U.S. Provisional Patent Application No. 63/ 802,001, filed on May 8, 2025, which application is incorporated by reference to the extent permitted by applicable law.

### FIELD OF THE INVENTION

The present invention relates to gas identification, and more particularly to a home detection system having a plurality of sensors for custom gas detection.

### BACKGROUND OF THE INVENTION

Gas detection and identification is critical within home environments. Special attention is directed to the detection of harmful gases, volatile organic compounds (VOCs), and airborne substances. Monitoring for harmful gases and substances serve various purposes such as personalized health monitoring and environmental checks, but also the need for safeguarding homes from harmful gas exposure. Home gas sensors may vary in size, design, materials, and operation. Therefore, a custom gas detection system is desired to monitor air quality and alert users of the presence of harmful substances.

### SUMMARY OF THE INVENTION

The present invention described herein is a gas detection system that includes a plurality of detectors, a server, a plurality of actuators, and a mobile application. The plurality of detectors receive ambient air and create a response pattern. The response pattern is transmitted to the server which includes a database and an identification module. The database includes a plurality of known response patterns that correspond to known gases, substances, and odors. The identification module translates the response pattern received from the detectors into a known gas identity. The plurality of actuators remotely open or close one or more gas valves. The mobile application remotely monitors and controls the gas detection system. The server is in electronic communication with the detectors, identification module, actuators, and mobile application.

### BRIEF DESCRIPTION OF THE DRAWINGS

The features of the exemplary embodiments of the present invention will be described with reference to the following drawings, where like elements are labeled similarly, and in which:

FIG. 1 is a schematic view of a gas detection system of the present disclosure;

FIG. 2 is an exploded view of a detector of the gas detection system from FIG. 1;

FIG. 3 is an exploded isometric view of the detector of the gas detection system from FIG. 1;

FIG. 4 is a front view of an actuator of the gas detection system from FIG. 1;

FIG. 5 is an exploded view of the actuator of the gas detection system from FIG. 1; and

FIG. 6 is a schematic diagram of the mobile application for the gas detection system of the present disclosure;

FIG. 7 is a schematic diagram of the identification module of the mobile application user for the gas detection system of the present disclosure;

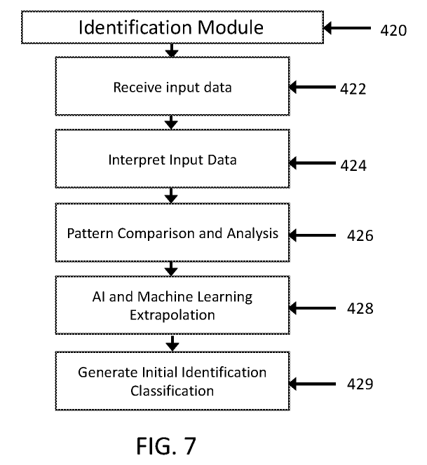

FIG. 8 is a schematic diagram of the validation module of the mobile application user for the gas detection system of the present disclosure; and

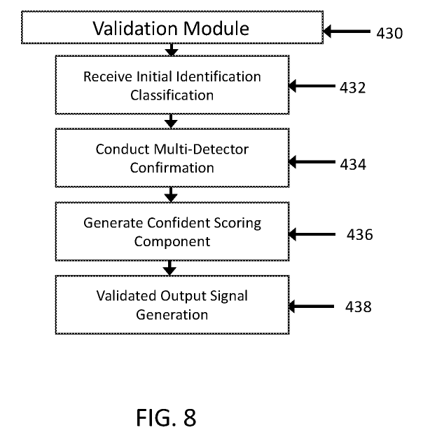

FIG. 9 is a graphical user interface of a mobile application for the gas detection system of the present disclosure.

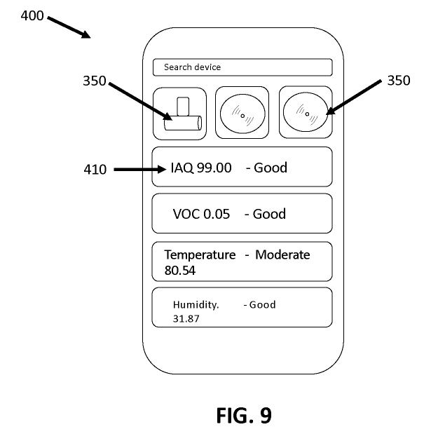

All drawings are schematic and not necessarily to scale. Parts given a reference numerical designation in one figure may be considered to be the same parts where they appear in other figures without a numerical designation for brevity unless specifically labeled with a different part number and described herein.

### DETAILED DESCRIPTION OF THE EMBODIMENTS

The features and benefits of the invention are illustrated and described herein by reference to exemplary embodiments. This description of exemplary embodiments is intended to be read in connection with the accompanying drawings, which are to be considered part of the entire written description. Accordingly, the disclosure expressly should not be limited to such exemplary embodiments illustrating some possible non-limiting combination of features that may exist alone or in other combinations of features.

In the description of embodiments disclosed herein, any reference to direction or orientation is merely intended for convenience of description and is not intended in any way to limit the scope of the present invention. Relative terms such as "lower," "upper," “horizontal,” “vertical,”, “above,” “below,” “up,” “down,” “top” and “bottom” as well as derivative thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise.

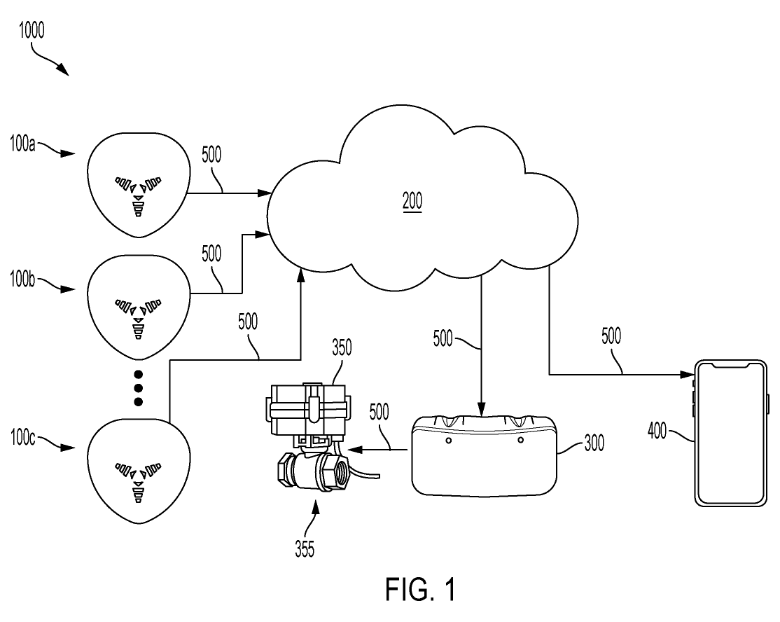

FIG. 1 depicts an exemplary embodiment of a gas detection system 1000 according to the present disclosure. Unlike traditional gas detection devices, which are limited to detecting only a few specific gases, and are not connected to a user's device for real time monitoring; the gas detection system 1000 allows for real time monitoring and detection of custom gas profiles and harmful substances. The gas detection system 1000 incorporates machine learning and artificial intelligence for more accurate detection and identification of harmful substances. Once a harmful substance is detected, the gas detection system can alert the user through a mobile application, and/or through email notification, and in some embodiments, the user can control an actuator to shut off certain gas valves to prevent further spread of the detected gases. For example, in some embodiments, the system 1000 includes a plurality of detectors, each programmed to detect a different substance, including but not limited to Carbon Monoxide, Methane, Butane, Nitrogen Oxides, VOCs, Smoke, Temperature, Humidity, and Pressure; the detectors then record the readings in the ambient air to create a unique pattern for each gas mixture. The reading is then compared to a database of known substances, gases, and odors, utilizing artificial intelligence to detect digital signatures of gases and substances, and then to classify the readings into various alert levels. In some embodiments, where the alert level is low, the system only logs the data into a database. In some embodiments, where the alert level is high, the system will begin to flash the LEDs, alarm a buzzer, and provide a push notification to a user's phone. In some embodiments, where the alert level is critical, the system will flash the LEDs, alarm the buzzer, provide a push notification to a user's phone, and activate a valve shut-off system to limit gases from entering the environment.

As briefly described above, in some embodiments, the gas detection system 1000 includes a plurality of detectors 100 that are in electronic communication 500 with a server 200, and an actuator 300. The server 200 is in electronic communication 500 with a mobile application 400 where a user can view and monitor each detector to observe a current reading 410, control the actuator 300 to shut off any gas lines that are active, and monitor room air quality, relative humidity, temperature, and volatile organic compounds (VOCs).

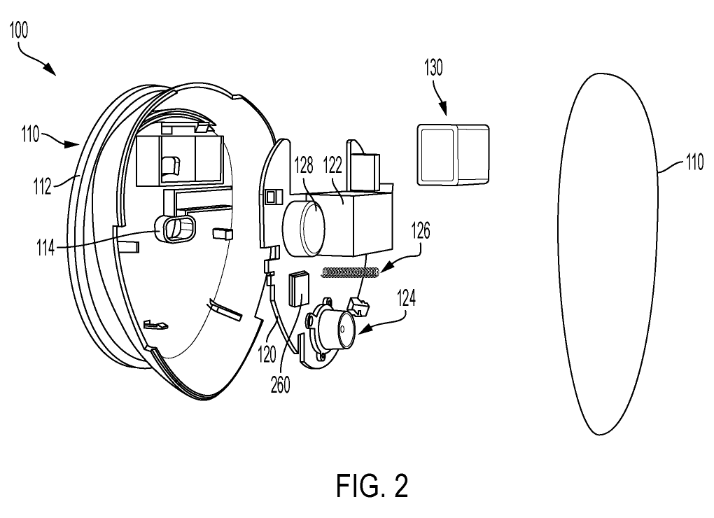

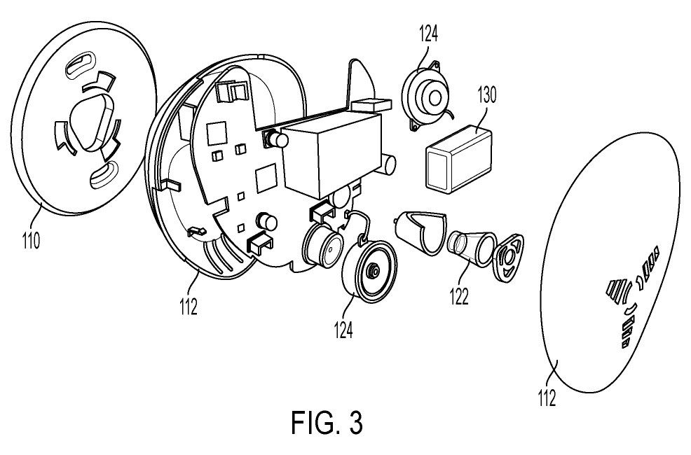

The detectors 100 are shown in further detail in FIG. 2. In some embodiments, as shown for example in FIGS. 2-3, the detectors 100 include a casing 110, a circuit board 120, and a power supply 130. The casing 110 includes a base member 112 having an inlet aperture 114 for allowing the circuit board 120 to be in fluid communication with the ambient air. The circuit board 120 includes a gas sensor 122 that is aligned with the inlet aperture 114 for being in fluid communication with the ambient air. When exposed to the ambient air, one or more gas sensors 122 generates a response pattern based on the substances within the air. In some embodiments, the response pattern generated from the gas sensors 122 is used to detect smoke, harmful gases, air quality, and personalized gases. In some embodiments, the harmful gases include carbon monoxide, nitrogen dioxide, hydrogen, methane, propane, ethanol and ammonia. However, as will be described in more detail later, the gas detector 100 can be trained to detect any number of gases and harmful substances through artificial intelligence and machine learning.

In some embodiments, the circuit board 120 further includes a notification system 124 for notifying a user about a detection of a harmful gas or increase in any harmful gas level within the ambient air. In some embodiments, the notification system 124 includes LED lights, and/or a horn or speaker to notify the user. In some embodiments, the detectors100 further includes a touch button 126 for silencing the notification system 124. In some embodiments, the circuit board 120 further includes a communication module 128 for communicating with server 200. In some embodiments, communication module 128 includes a WiFi adapter. In some embodiments, communication module 128 includes a Bluetooth adapter. In some embodiments, the gas detection system 1000 includes more than one detector 100. In some embodiments, the gas detection system 1000 is used for residential homes and includes a plurality of detectors 100 located in each of the rooms within the residential home. In some embodiments, the gas detection system 1000 is used for commercial buildings. In some embodiments, the detectors 100 create response patterns at pre-determined intervals. For example, every second ambient air is tested by the detector. In some embodiments, the detectors 100 create response patterns at more spaced apart intervals such as every 3-5 minute. In some embodiments, the detectors 100 monitor ambient air in real-time.

In some embodiments, the power supply 130 includes a main line that is powered through VAC, and the power supply 130 includes a backup line that is powered through a rechargeable DC battery. In some embodiments, the rechargeable DC battery backup is a replaceable 9V battery.

Detectors 100 are in electronic communication 500 with server 200. In some embodiments, server 200 is a cloud server that is coordinated with each of the plurality of detectors 100, which allows the receipt and processing of response patterns from detectors 100. Server 200 processes the response patterns from detectors 100 to identify the presence of gases and harmful substances, including gases such as smoke, carbon monoxide, nitrogen dioxide, ethanol, hydrogen, ammonia, methane, propane, butane, volatile organic compounds (VOCs), and NOx. In some embodiments, server 200 processes response patterns from detectors 100 to identify temperature, humidity, pressure, and air quality. In some embodiments, server 200 includes data storage for housing historical measurements and response patterns, previous alerts and warning, and housing configuration. In some embodiments, server 200 includes an identification module 250 for analyzing the response pattern data being received by detectors 100. In some embodiments, identification module 250 utilizes machine learning for more accurate readings and identification of gases and harmful substances through past identifications. In some embodiments, identification module 250 utilizes artificial intelligence for expanding the detection and identification scope for the gas detection system 1000. The combination of machine learning and artificial intelligence allows for the gas detection system 1000 to be trained to detect more gases as more gas profiles become available. For example, in some embodiments, the gas detection system can be trained to identify a specific gas when certain response patterns are detected. In some embodiments, the gas detection system 1000 may be remotely updated to detect more gases and harmful substances through Firmware updates that further train the identification module 250. In some embodiments, the gas detection system 1000 is trained to detect carbon monoxide (CO), Methane (CH₄), Propane (C₃H8), Butane (C₄H10), Nitrogen Oxides (NOₓ), Volatile Organic Compounds (VOCs), and Smoke. In some embodiments, the system can detect Carbon Monoxide at a reading range between 0 – 1,000ppm and configured to alert if the system detects a reading greater than 10ppm. In some embodiments, the system can detect Methane at a reading range between 0 – 5,000ppm and configured to alert if the system detects a reading greater than 1000ppm. In some embodiments, the system can detect Propane at a reading range between 0 – 5,000ppm and configured to alert if the system detects a reading greater than 1,500ppm. In some embodiments, the system can detect Butane at a reading range between 0 – 5,000 ppm and configured to alert if the system detects a reading greater than 500ppm. In some embodiments, the system can detect Nitrogen Oxides at a reading range between 0 – 2,000ppb and configured to alert if the system detects a reading greater than 150ppm. In some embodiments, the system can detect VOCs at a reading range between 0 – 2,000ppb and configured to alert if the system detects a reading greater than 100ppb. In some embodiments, the system can detect Smoke and configured to alert if the system detects its presence.

In some embodiments, the identification module 250 generates an identification classification based on the comparison between the received response pattern and the plurality of known response patterns stored in the database. The identification classification indicates the detected substance, the estimated concentration level, and a confidence level associated with the identification. In some embodiments, the identification classification includes metadata such as the detector location, timestamp, and environmental conditions at the time of detection. The identification classification serves as the input for subsequent validation processing to determine whether a response action should be initiated.

In some embodiments, the gas detection system 1000 includes a validation module 260 configured to receive the identification classification from the identification module 250 and validate the identification classification according to one or more validation criteria prior to initiating a response action. The validation module 260 is a critical safety component that prevents false positive actuations and ensures that response actions such as valve closure are only initiated when the detection is confirmed to be accurate and reliable. The validation module 260 generates a validated output only when the identification classification satisfies the one or more validation criteria. By requiring validation before actuation, the system 1000 reduces nuisance alarms, prevents unnecessary valve closures that could disrupt normal operations, and improves overall system reliability and user confidence.

In some embodiments, the one or more validation criteria applied by the validation module 260 include threshold concentration level validation that requires the identification classification to indicate a detected concentration level exceeding a predetermined threshold before a validated output is generated. For example, the validation module 260 may require that carbon monoxide concentration exceed 10ppm, methane concentration exceed 1000ppm, or propane concentration exceed 1500ppm before authorizing actuation of valves 355. In some embodiments, the validation module 260 applies temporal validation that requires the identification classification to persist for a minimum duration before the validated output is generated. For example, the validation module 260 may require that the detected substance remain above the threshold concentration for at least 5 seconds, 10 seconds, or 30 seconds to filter out transient spikes or momentary sensor anomalies. This temporal validation prevents false actuations caused by brief environmental fluctuations or sensor noise.

In some embodiments, the validation module 260 applies multi-detector confirmation as a validation criterion, requiring at least two detectors from the plurality of detectors 100 to generate response patterns corresponding to matching identification classifications before a validated output is generated. This cross-validation approach provides spatial confirmation that a detected substance is present in multiple locations, reducing the likelihood that a single faulty sensor or localized anomaly will trigger an unnecessary response action. For example, if detectors 100a in the kitchen detects high methane concentration but detector 100b in the adjacent utility room does not detect elevated methane, the validation module 260 may withhold the validated output pending further confirmation.

In some embodiments, the validation module 260 is configured to assign a confidence score to the identification classification, and the validated output is generated only when the confidence score exceeds a predetermined confidence threshold. The confidence score may be based on factors such as signal strength from the gas sensors 122, consistency of the response pattern with known patterns in the database, agreement between multiple sensors within a single detector 100, and historical accuracy of the particular detector 100. For example, the validation module 260 may assign a confidence score on a scale of 0 to 100 and may require a confidence score of at least 80 before generating the validated output. In some embodiments, the confidence threshold varies based on the severity of the detected substance, with lower confidence thresholds applied for highly hazardous substances such as carbon monoxide and higher confidence thresholds applied for less critical substances.

In some embodiments, the validation module 260 comprises a machine learning algorithm trained on historical sensor data to distinguish between valid detection events and false positive events. The machine learning algorithm is applied to the identification classification prior to generating the validated output. The machine learning algorithm may be trained using supervised learning on a dataset comprising thousands of historical detection events labeled as either valid detections or false positives. Features used by the machine learning algorithm may include the shape of the response pattern over time, the rate of concentration increase, environmental conditions such as temperature and humidity, time of day, recent system activity, and patterns of previous detections. By learning from historical data, the machine learning algorithm can identify subtle indicators of false positives that may not be captured by simple threshold rules, thereby improving the accuracy of the validation process.

In some embodiments, the validation module 260 applies environmental cross-validation that compares the identification classification against at least one environmental parameter selected from temperature, humidity, pressure, and time of day. Certain gases and substances exhibit detection patterns that correlate with environmental conditions, and cross-validation against these parameters can help confirm or refute a detection. For example, if the identification classification indicates detection of volatile organic compounds (VOCs) but the temperature and humidity are within normal ranges and there has been no recent activity in the environment, the validation module 260 may flag the detection as potentially anomalous and withhold the validated output pending additional confirmation. Conversely, if VOCs are detected during cooking hours when temperature and humidity are elevated, the detection may be validated as consistent with expected environmental conditions.

In some embodiments, the validation module 260 is configured to require manual user confirmation via the mobile application 400 before generating the validated output when the identification classification indicates a detected substance having a risk level below a predetermined risk threshold. For low-risk or non-hazardous substances, the validation module 260 may present the identification classification to the user through the mobile application 400 and request confirmation before proceeding with actuation of valves 355 or other response actions. This user-in-the-loop approach provides flexibility for situations where automated actuation may be undesirable, while still allowing the user to quickly confirm and authorize the response action if appropriate. For high-risk substances such as carbon monoxide or natural gas leaks, the validation module 260 may bypass user confirmation and proceed directly with automated response actions to ensure rapid mitigation of the hazard.

The server 200 is also in electronic communication 500 with one or more actuators 300. Actuators 300 are communicatively coupled to the validation module 260 and act as controllers for one or more responses devices 350 such that the response devices 350 can activated or controlled remotely and/or in person. In some embodiments, the response devices 350 include valves configured to open or close in response to receiving the validated output from the validation module 260. By requiring a validated output before actuation, the system 1000 ensures that response devices 350 are only actuated when a genuine hazard has been confirmed through the validation process, thereby preventing unnecessary disruptions to normal operations. In some embodiments, the response devices 350 include alarms configured to emit audible or visual warnings. In some embodiments, the response devices 350 include ventilation systems configured to activate air circulation or exhaust systems. In some embodiments, the response devices 350 include notification systems configured to send alerts to emergency services or building management. In some embodiments, the response devices 350 include shutoff devices configured to terminate power supply or other utility connections. In some embodiments, the response devices 350 include locks configured to secure or restrict access to affected areas. In some embodiments, multiple response devices 350 of different types are activated simultaneously based on the validated output from the validation module 260.

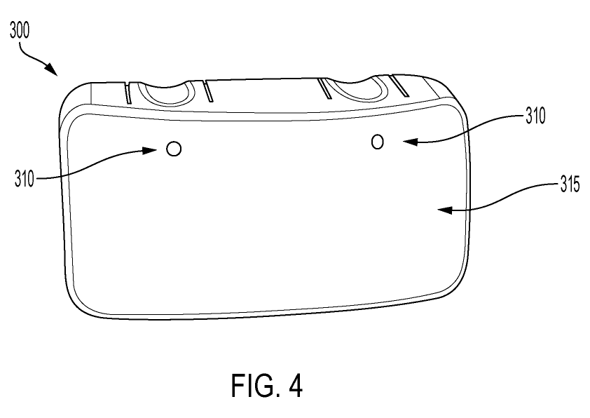

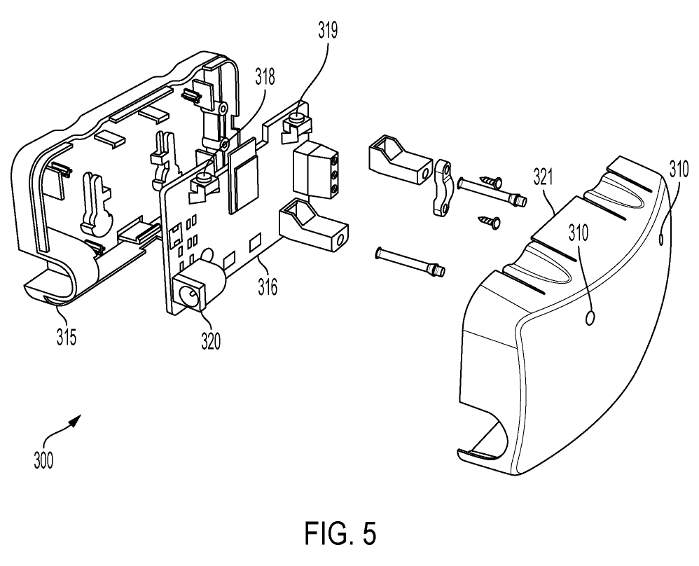

As shown in FIGS. 4-5, Actuator 300 has a housing 321 that includes a base 315, an electronic board 316, a button 318 to configure the WiFi network, a button 319 for manual control related to the activation of each of the one or more response devices 350 through the terminal block 320. In some embodiments, the response devices 350. In some embodiments, the actuator is connected with WiFi connectivity for electronic communication 500 with the server 200. In other embodiments, the actuator 300 is connected with Bluetooth or infrared for electronic communication 500 with server 200. In some embodiments, the actuator has a physical button for the user to manually actuating the response devices 350. Similar to the detectors 100, in some embodiments, actuator 300 may be powered through VAC, and actuator 300 includes a backup line that is powered through a rechargeable DC battery. In some embodiments, the rechargeable DC battery backup is a replaceable 9V battery. In some embodiments, activation of the response devices is controlled by the actuator is automatically controlled based on the validated output provided from the validation module 260. For example, when the presence of a gas leak is detected by gas detectors 100, the identification module 250 generates an identification classification, the validation module 260 validates the identification classification according to the validation criteria, and upon satisfaction of the validation criteria, the validation module 260 generates a validated output that is transmitted to the actuator 300, causing the actuator 300 to close any open valves 355. In some embodiments, the actuator 300 includes one or more LED lights 310 to signal that WiFi communication is active, the valves 355 are open, or valves 355 are closed.

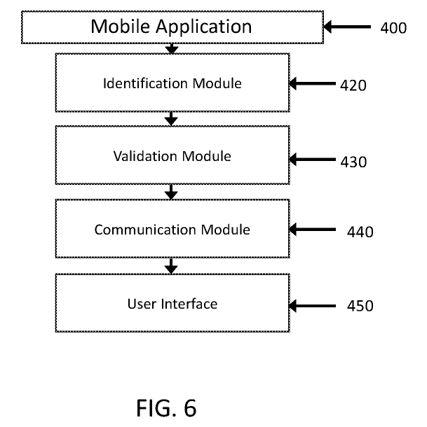

In some embodiments, the gas detection system 1000 includes a mobile application 400 that provides a graphical user interface (GUI) for the user to monitor the gas detection system 1000 (FIG. 6). In some embodiments, the mobile application 400 is configured to provide remote monitoring and control of the gas detection system 1000, wherein the mobile application 400 displays sensor data, identification results from the identification module 250, and validation status from the validation module 260. The mobile application provides a summary view for the user such that the user can check the real-time readings from each detectors100, observe measurements regarding the real-time relative humidity, air quality, temperature, and historical readings for the detectors 100. In some embodiments, the validation status displayed by the mobile application 400 includes information such as whether the current identification classification has passed or failed validation criteria, which specific validation criteria have been satisfied, the confidence score assigned to the identification classification, and whether a validated output has been generated. This validation status information provides transparency to the user regarding the system's decision-making process and helps build user confidence in the reliability of the system. In some embodiments, the mobile application 400 also includes a control feature for remotely controlling the actuator 300 such that the attached valves 355 can be opened or closed depending on the user's input. In some embodiments, the control feature also includes an option for remotely silencing or dismissing the notification system while the notification system 124 is active. In some embodiments, the mobile application 400 also includes an AI library for downloading and updating the user's gas detection system 1000 to include additional classifications of new gases or odors such as, for example, coffee, limonene, cannabis, rotten meat, alcohol, and other such household gases and odors.

In some embodiments, the gas detection system 1000 conducts the following series of tasks for detecting the presence of harmful gases in the ambient air. First, detectors 100 receive a quantity of ambient air through the inlet aperture 114. Then, the ambient air contacts the one or more gas sensors 122 and a response pattern is produced. The communication module 128 then transmits the response pattern to the server 200. Server 200 receives response pattern and identification module 250 begins to process the response pattern to compare the response pattern against the existing database of known gases, odors, and harmful substances that are known in the server 200. Machine learning and artificial intelligence will then extrapolate the readings to generate an identification classification. The identification classification is then transmitted to the validation module 260, which validates the identification classification according to one or more validation criteria prior to initiating a response action. The validation module 260 applies validation criteria such as threshold concentration validation, temporal validation, multi-detector confirmation, confidence scoring, machine learning-based false positive detection, and environmental cross-validation. If the identification classification satisfies the one or more validation criteria, the validation module 260 generates a validated output. If the validated output indicates that a harmful substance has been detected and the substance exceeds a maximum allowable threshold, the server will communicate with the notification system 124 to activate an alarm to notify the user that a high concentration of harmful substance is present. The server 200 will also send an alert to the user through the mobile application 400, including the validation status and confidence level associated with the detection. In some embodiments, such as for example when a given substance is detected within a critical range and the validation criteria are satisfied, the validation module 260 generates a validated output that is transmitted to the actuator 300, causing the actuator 300 to close any open valves 355 to prevent the concentration of harmful substance from increasing within the measured area. The user will then take any necessary precautions based on the identified substance, and the user can silence the notification system 124 if necessary. Once completed, the response pattern and validation results will be stored in the database of the server 200 such that the machine learning module can utilize the previous response pattern and validation outcomes for future identifications and validation processing.

In some embodiments, the gas detection system 1000 is implemented as part of a broader home automation platform. In such embodiments, a central hub is provided that communicates wirelessly with a plurality of sensors and a plurality of electronic devices. The plurality of sensors are wirelessly coupled to the central hub and include the gas detectors 100 described above as well as additional sensors configured to detect environmental conditions such as temperature, humidity, air quality, particulate matter, carbon dioxide levels, and occupancy. Each sensor is configured to detect an environmental condition and generate a response pattern. The plurality of electronic devices wirelessly paired to the central hub include at least one controllable device selected from thermostats, ventilation systems, valves, alarms, locks, lighting systems, and climate control systems. The central hub includes an identification module configured to analyze the response pattern from the plurality of sensors and identify a detected condition by comparing the response pattern to a library of known patterns stored in a database. A validation engine within the central hub receives the response pattern and identification results from the identification module, applies a validation process to the response pattern to determine whether the sensor data meets predetermined validation criteria, and authorizes a control action when the sensor data meets the predetermined validation criteria. A control module communicatively coupled to the validation engine is configured to actuate one of the plurality of electronic devices upon authorization from the validation engine, wherein the control module selects which electronic device to actuate based on the identified detected condition and a set of predefined response rules. For example, if the detected condition corresponds to elevated carbon dioxide levels, the control module may select and actuate a ventilation system to increase fresh air circulation. If the detected condition corresponds to detection of a gas leak, the control module may select and actuate a valve to shut off gas supply. If the detected condition corresponds to a temperature anomaly, the control module may select and actuate a thermostat to adjust heating or cooling. A user interface accessible via a mobile application provides the user with the ability to display real-time sensor data, condition identification results, validation status, and operational status of the plurality of electronic devices.

In some embodiments, each detector 100 comprises a multi-sensor array including at least two different sensor types, and the response pattern is generated based on combined outputs from the at least two different sensor types. For example, a single detectors 100 may include a metal oxide semiconductor (MOS) gas sensor for detecting combustible gases, an electrochemical sensor for detecting carbon monoxide, a particulate sensor for detecting smoke, a temperature sensor, and a humidity sensor. By combining outputs from multiple sensor types, the detector 100 can generate a more distinctive and reliable response pattern that improves the accuracy of the identification module 250 and reduces the likelihood of false positives. The multi-sensor array approach also enables the detectors 100 to simultaneously detect multiple substances and environmental conditions, providing a comprehensive assessment of air quality and safety.

In some embodiments, the predetermined validation criteria applied by the validation engine in the home automation platform include threshold validation that compares the sensor data to a predetermined threshold value, and temporal validation that requires the sensor data to persist above the predetermined threshold value for a minimum time period. In some embodiments, the validation engine is configured to perform cross-sensor correlation by comparing response patterns from at least two sensors positioned in different locations, and the validation engine authorizes the control action only when the response patterns from the at least two sensors both correspond to detection of a same environmental condition. This cross-sensor correlation provides spatial confirmation of the detected condition and reduces false positives caused by localized anomalies or sensor malfunctions.

In some embodiments, the predefined response rules implemented by the control module include a cascading response protocol comprising a first-level response action followed by a second-level response action, wherein the second-level response action is initiated when the sensor data continues to meet the predetermined validation criteria after a predetermined time period following execution of the first-level response action. For example, the first-level response action may comprise activating a ventilation system from the plurality of electronic devices to attempt to reduce the concentration of the detected substance through increased air circulation, and the second-level response action may comprise closing a valve from the plurality of electronic devices if the ventilation system does not successfully reduce the concentration below acceptable levels within the predetermined time period. This cascading approach provides a graduated response that attempts less disruptive mitigation measures before resorting to more severe actions such as valve closure.

In some embodiments, the control module is configured to implement intelligent device selection based on the type of detected condition. For example, the control module may be configured to select a thermostat from the plurality of electronic devices when the identified detected condition corresponds to a temperature anomaly, select a ventilation system from the plurality of electronic devices when the identified detected condition corresponds to an air quality anomaly, and select a valve from the plurality of electronic devices when the identified detected condition corresponds to detection of a hazardous substance. This condition-specific device selection ensures that the most appropriate response action is taken for each type of detected condition.

In some embodiments, the validation engine is configured to apply adaptive validation thresholds that vary based on at least one contextual factor selected from time of day, detected occupancy status within the environment, and a user-selected operational mode. For example, during nighttime hours when occupants are sleeping, the validation engine may apply lower validation thresholds to provide more sensitive detection and faster response to potential hazards. During daytime hours when normal activities such as cooking may generate temporary gas emissions, the validation engine may apply higher validation thresholds to reduce nuisance alarms. Similarly, when the system detects that the home is unoccupied (for example, based on input from motion sensors or smart locks), the validation engine may apply more conservative validation thresholds and authorize more aggressive response actions such as immediate valve closure. When the system is in a user-selected "home" mode indicating that occupants are present, the validation engine may apply validation thresholds that balance safety with avoiding unnecessary disruptions to normal activities.

While the foregoing description and drawings represent exemplary embodiments of the present disclosure, it will be understood that various additions, modifications and substitutions may be made therein without departing from the spirit and scope and range of equivalents of the accompanying claims. In particular, it will be clear to those skilled in the art that the present invention may be embodied in other forms, structures, arrangements, proportions, sizes, and with other elements, materials, and components, without departing from the spirit or essential characteristics thereof. In addition, numerous variations in the methods/processes described herein may be made within the scope of the present disclosure. One skilled in the art will further appreciate that the embodiments may be used with many modifications of structure, arrangement, proportions, sizes, materials, and components and otherwise, used in the practice of the disclosure, which are particularly adapted to specific environments and operative requirements without departing from the principles described herein. The presently disclosed embodiments are therefore to be considered in all respects as illustrative and not restrictive. The appended claims should be construed broadly, to include other variants and embodiments of the disclosure, which may be made by those skilled in the art without departing from the scope and range of equivalents.

What is claimed is:

1. A gas detection system comprising:
a plurality of detectors, each detector including one or more sensors configured to generate a response pattern when the one or more sensors contact ambient air;
a server including:
a database storing a plurality of known response patterns, each known response pattern corresponding to at least one known gas, substance, or odor; and
an identification module configured to compare the response pattern generated by the plurality of detectors with the plurality of known response patterns stored in the database to generate an identification classification;
a validation module configured to:
receive the identification classification;
validate the identification classification according to one or more validation criteria prior to initiating a response action; and
generate a validated output only when the identification classification satisfies the one or more validation criteria;
a plurality of actuators communicatively coupled to the validation module, each actuator configured to control at least one response device in response to receiving the validated output from the validation module; and
a mobile application configured to provide remote monitoring and control of the gas detection system, wherein the mobile application displays sensor data, identification results from the identification module, and validation status from the validation module.

2. The gas detection system of claim 1, wherein the one or more validation criteria include a threshold concentration level that requires the identification classification to indicate a detected concentration level exceeding a predetermined threshold.

3. The gas detection system of claim 1, wherein the one or more validation criteria include temporal validation that requires the identification classification to persist for a minimum duration before the validated output is generated.

4. The gas detection system of claim 1, wherein the one or more validation criteria comprise multi-detector confirmation that requires at least two detectors from the plurality of detectors to generate response patterns corresponding to matching identification classifications.

5. The gas detection system of claim 1, wherein the validation module is configured to assign a confidence score to the identification classification, and wherein the validated output is generated only when the confidence score exceeds a predetermined confidence threshold.

6. The gas detection system of claim 1, wherein the validation module comprises a machine learning algorithm trained on historical sensor data to distinguish between valid detection events and false positive events, and wherein the machine learning algorithm is applied to the identification classification prior to generating the validated output.

7. The gas detection system of claim 1, wherein the one or more validation criteria comprise environmental cross-validation that compares the identification classification against at least one environmental parameter selected from temperature, humidity, pressure, and time of day.

8. The gas detection system of claim 1, wherein each detector comprises a multi-sensor array including at least two different sensor types, and wherein the response pattern is generated based on combined outputs from the at least two different sensor types.

9. The gas detection system of claim 1, wherein the plurality of known response patterns stored in the database correspond to at least one substance selected from natural gas, carbon monoxide, carbon dioxide, propane, methane, hydrogen sulfide, ammonia, volatile organic compounds, smoke particulates, and combustible gases.

10. The gas detection system of claim 1, wherein the system operates autonomously without network connectivity.

11. The gas detection system of claim 1, wherein the at least one response device comprises at least one device selected from the group consisting of valves, alarms, ventilation systems, notification systems, shutoff devices, locks, and climate control systems.

12. A home automation platform comprising:
a central hub;
a plurality of sensors wirelessly coupled to the central hub, each sensor configured to detect an environmental condition and generate a response pattern;
a plurality of electronic devices wirelessly paired to the central hub, the plurality of electronic devices including at least one controllable device selected from thermostats, ventilation systems, valves, alarms, locks, lighting systems, and climate control systems;
an identification module configured to analyze the response pattern from the plurality of sensors and identify a detected condition by comparing the response pattern to a library of known patterns stored in a database;
a validation engine configured to:
receive the response pattern and identification results from the identification module;
apply a validation process to the response pattern to determine whether the sensor data meets predetermined validation criteria; and
authorize a control action when the response pattern meets the predetermined validation criteria;
a control module communicatively coupled to the validation engine and configured to actuate one of the plurality of electronic devices upon authorization from the validation engine,
wherein the control module selects which electronic device to actuate based on the identified detected condition and a set of predefined response rules; and
a user interface accessible via a mobile application, the user interface configured to display real-time sensor data, condition identification results, validation status, and operational status of the plurality of electronic devices.

13. The home automation platform of claim 12, wherein the predetermined validation criteria include threshold validation that compares the sensor data to a predetermined threshold value, and temporal validation that requires the sensor data to persist above the predetermined threshold value for a minimum time period.

14. The home automation platform of claim 12, wherein the validation engine is configured to perform cross-sensor correlation by comparing response patterns from at least two sensors positioned in different locations, and wherein the validation engine authorizes the control action only when the response patterns from the at least two sensors both correspond to detection of a same environmental condition.

15. The home automation platform of claim 12, wherein the predefined response rules include a cascading response protocol comprising a first-level response action followed by a second-level response action, and wherein the second-level response action is initiated when the sensor data continues to meet the predetermined validation criteria after a predetermined time period following execution of the first-level response action.

16. The home automation platform of claim 15, wherein the first-level response action comprises activating a ventilation system from the plurality of electronic devices, and wherein the second-level response action comprises closing a valve from the plurality of electronic devices.

17. The home automation platform of claim 12, wherein the control module is configured to select a thermostat from the plurality of electronic devices when the identified detected condition corresponds to a temperature anomaly, select a ventilation system from the plurality of electronic devices when the identified detected condition corresponds to an air quality anomaly, and select a valve from the plurality of electronic devices when the identified detected condition corresponds to detection of a hazardous substance.

18. The home automation platform of claim 12, wherein the validation engine is configured to apply adaptive validation thresholds that vary based on at least one contextual factor selected from time of day, detected occupancy status within the environment, and a user-selected operational mode.

19. The home automation platform of claim 12, wherein the plurality of sensors includes at least one sensor selected from the group consisting of chemical gas sensors, air quality sensors, particulate matter sensors, temperature sensors, humidity sensors, carbon monoxide sensors, carbon dioxide sensors, smoke detectors, and volatile organic compound sensors.

20. The home automation platform of claim 12, wherein the platform operates autonomously without network connectivity.
